(19) United States
(12) Patent Application Publication
(10) Pub. No.: US 2021/0355699 A1
(43) Pub. Date: Nov. 18, 2021

(54) SYSTEM FOR SANITIZING AND CONTROLLING PEOPLE WHO WANT TO ACCESS A ROOM

(71) Applicant: FARAM TECH LAB s.r.l., Giavera del Montello (IT)

(72) Inventor: Paolo RIGHETTO, Giavera del Montello (IT)

(21) Appl. No.: 16/911,462

(22) Filed: Jun. 25, 2020

(30) Foreign Application Priority Data
May 18, 2020 (IT) 20 2020 000002611

(51) Int. Cl. E04H 1/12; G09F 25/00; F24F 3/16; A61L 2/24; A61L 2/22; G07C 9/00; G05B 19/4155; G06K 9/00

(52) U.S. Cl. CPC E04H 1/12; G09F 25/00; F24F 3/16; A61L 2/24; A61L 2/22; G07C 9/00174; G05B 2219/45078; G06K 9/00288; G09F 2025/005; A61L 2202/122; A61L 2202/121; A61L 2202/14; A61L 2202/15; G05B 19/4155

(57) ABSTRACT

A system for sanitizing and controlling people who want to access a room (40), adapted to at least reduce the risk that people can bring pathogenic contaminants into said room (40), in particular viral pathogenic contaminants, such as for example coronavirus,

comprising: a sanitizing cabin (1) having a size such that it can house at least one person inside; a sanitizing unit (7) for sanitizing the person present inside said cabin, and at least one controller (12) which is operatively connected to a sanitation activating element (32, 36, 100E), said controller (12) being configured to determine a process control command for said sanitizing unit (7) to supply a sanitizing substance to said cabin for a predetermined time, when it receives at least one activation signal from said sanitation activating element (32, 36, 100E).

The disclosure of Italian Utility Model Application No. 20 2020 00000 2611, filed May 18, 2020, is incorporated herein by reference.

### FIELD OF THE INVENTION

Object of the present invention is a system for sanitizing and controlling people who want to access a room, adapted to at least reduce the risk that people can bring pathogenic contaminants into said room, according to the characterizing part of the main claim.

Sterilization devices for sterilizing people who have to access cleanrooms of industrial plants (such as, for example, plants for manufacturing microprocessors, or plants of chemical industries) or sterile environments for example particular hospital environments have long been known. People who use these devices must wear special protective clothing before undergoing sterilization treatments; these know devices have therefore had so far, a very limited and specific use. Moreover, because of the use they are intended for, these known devices must be made with extreme care and precision and using special materials, which complicates and lengthens the manufacturing and maintenance times and significantly affects their final cost.

The known devices are also generally fixed systems incorporated into the building that has a clean or sterile room, and are also generally bulky and difficult to transport.

In addition, they generally require specialized personnel for being operated.

### SUMMARY

The object of the present invention is to provide a system for sanitizing and controlling people who want to access to a room, adapted to at least reduce the risk that people can bring pathogenic contaminants into said room, which is easy and simple to implement and allows fast automatic sanitization of people who want to access to any room where people are expected to be present, such as a shop, a supermarket, a public office or a bank.

In this context, sanitizing is to be understood as a process capable of at least partially reducing pathogenic microorganisms of the viral type, on inanimate objects such as, for example, clothes worn by a person and/or on the person him/herself.

A further object is to provide a sanitization and control system that is simple to assemble and which is also easily transportable, and which can be easily placed in correspondence with the entrances of pre-existing rooms.

A further object is to provide a sanitization and control system that automatically prevents people with symptoms typical of a given pathology, in particular a covid-19 type viral pathology, from accessing to the room.

A further object is to provide a sanitization and control system that automatically prevents people who are not adequately protected with respect to the potential for contagion of a given pathology, in particular a covid-19 type viral pathology, from accessing to the room.

A further object is to provide a sanitization and control system that allows to automatically provide the person who has to undergo disinfection treatment with information about the treatment itself and/or any additional operations that this person must perform, and/or any further information of interest to said person.

A further object is to provide a sanitization and control system that prevents, or in any case limits, undesirable behaviours by the person who has to undergo the disinfection process.

A further object is to provide a sanitization and control system that allows to gather and process data on the people undergoing the disinfection treatment.

A further object is to provide a sanitization and control system that allows to automatically regulate the access of people to the disinfection treatment even on the basis of the data collected in the room a person wants to access to. These and other objects that will be apparent to one skilled in the art are achieved by a sanitization and control system according to the characterizing part of the attached claims.

### BRIEF DESCRIPTION OF THE DRAWING FIGURE

For a better understanding of the invention there are attached, by way of non-limiting example, the following drawings, wherein:

FIG. 1 shows a schematic perspective view of a system component according to the invention;

FIG. 2 shows a front schematic view thereof;

FIG. 3 shows a schematic sectional view thereof taken along line 3/3 of FIG. 2;

FIG. 4 shows a schematic sectional view thereof taken along line 4/4 of FIG. 2;

FIG. 5 shows a left side schematic view thereof;

FIGS. 6 and 7 show cross sectional schematic views of the system wherein two possible uses of the cabin are represented;

FIG. 8 shows a schematic view of a further system component;

FIGS. 9A and 9B show, through a block diagram, a first and a second control and command unit of the system;

FIG. 10 shows a perspective schematic view of a plurality of further components of the system;

FIG. 11 shows a perspective schematic view of the component of FIG. 1 when it is associated with a place to be accessed to after using the system;

FIGS. 12A and 12B show a schematic perspective view and a sectional view taken along line 12B of FIG. 12A of a further embodiment of the component of FIG. 1;

FIGS. 13A-J show schematic algorithms of particular operating phases of the system.

### DETAILED DESCRIPTION

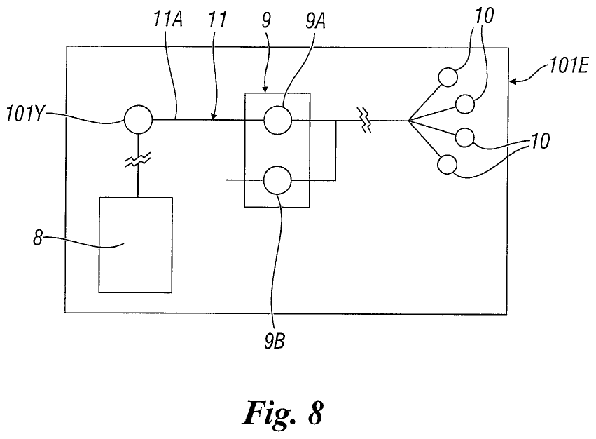

With reference to the aforementioned figures, they show a sanitization and control system for sanitizing and controlling people who want to access to a room, adapted to at least reduce the risk that people can bring pathogenic contaminants into said room, in particular viral pathogenic contaminants, such as for example coronavirus, comprising:
a sanitizing cabin 1 having a size such that it can house at least one person inside, comprising:
   at least one upper wall 2, one lower wall 3 and at least one side wall 4A-D (in the construction example shown in the figures there are four walls, the cabin having a parallelepiped shape),
   wherein said walls define a closed space L to which the access is possible through an entrance door 5 and from which it is possible to get out through an exit door 6, provided in the said walls in said at least one side wall,
a sanitizing unit 101E (schematically shown in FIG. 8) for sanitizing the person present inside said closed space L, comprising:
   a tank 8 of a sanitizing substance,
   a circuit 11 for conveying said substance from the tank to a plurality of emission members 10 emitting the substance in said closed room,
   and at least one member 9 for automatically conveying said substance from said tank to said emission members through said supply circuit,
at least one controller 12 configured to control said sanitizing unit 101E, to supply the sanitizing substance to said closed space L, when it receives one activation signal S1 from at least one sanitation activating element 100E, which is operatively connected to said controller 12, and when one person is present inside said closed room.

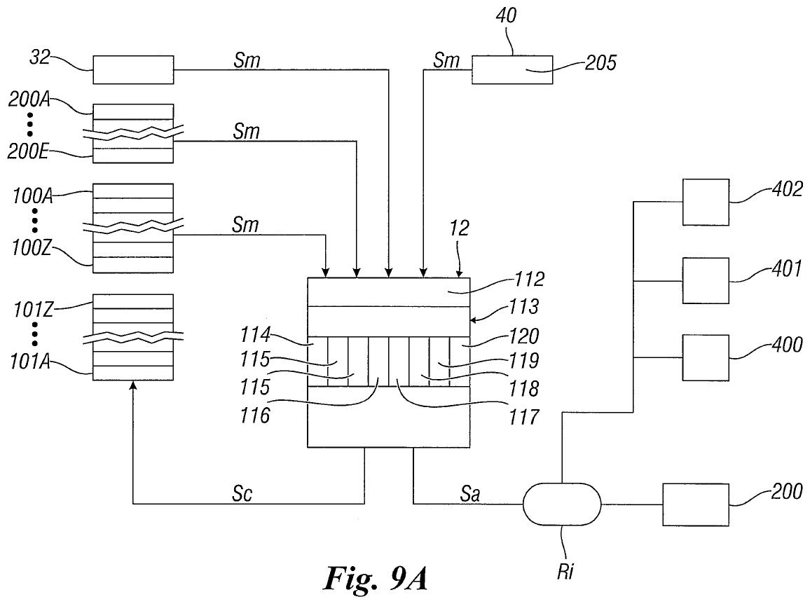

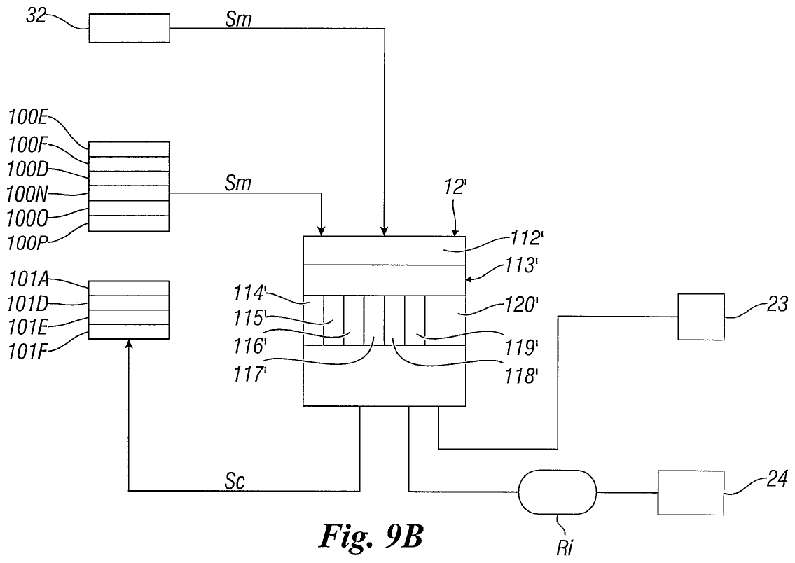

With reference to the controller 12, it is of a conventional type and will not therefore be described in detail below; it is represented in schematic view in FIG. 9A and is of the type comprising:
a known controller component 112 (for example a communication interface), adapted to receive a plurality of first monitoring signals Sm (described in detail below) from detecting members 100A-100N; 200A-E (also described in detail below),
a known controller component 113 for processing and evaluating monitoring the signals Sm (for example of the type adapted to classify and recognize the received signals Sm and determine a process command) adapted to generate a plurality of control signals Sc leaving the unit 12 for one or more of control members 1001A-101N provided in the cabin, as it will be described more in detail below.

The processing controller component 113 also advantageously provide a further controller component 114, for sending an alarm signal Sa, for example via an internet or a cellular telephone network Ri (schematically shown in FIG. 9A), to a remote supervision device 200, and/or external devices 400-402, they, too, described in detail below.

The controller 12, can be implemented in different ways, however all known to one skilled in the art. Preferably it is implemented through a controller using artificial intelligence techniques and one or more neural networks, for example of the type described in the Italian patent application IT201800009442A in the name of the Italian company Laser Navigation, incorporated by reference in the present text, (or also of the type described in U.S. Pat. No. 8,036,425 or WO2018/114443, they, too, incorporated by reference in the present text). Alternatively, the controller 12 can be implemented through a microprocessor device (for example a usual PC) which provides one or more computer programs adapted to implement the technical functions described below.

Even the remote supervision device 200 is of the type known to one skilled in the art; it is preferably connected to a plurality of systems according to the invention, and is adapted to manage the alarm signals coming from these systems, so that qualified personnel can intervene to solve the problem that has generated the alarm. This remote device, in a simplified version thereof, can for example be a smartphone and the alarm signal can be a message received on said phone.

As discussed below, the system could also provide two separate controllers, for example one for controlling and operating the sanitization and one for controlling and operating the temperature measurement and the presence of the mask.

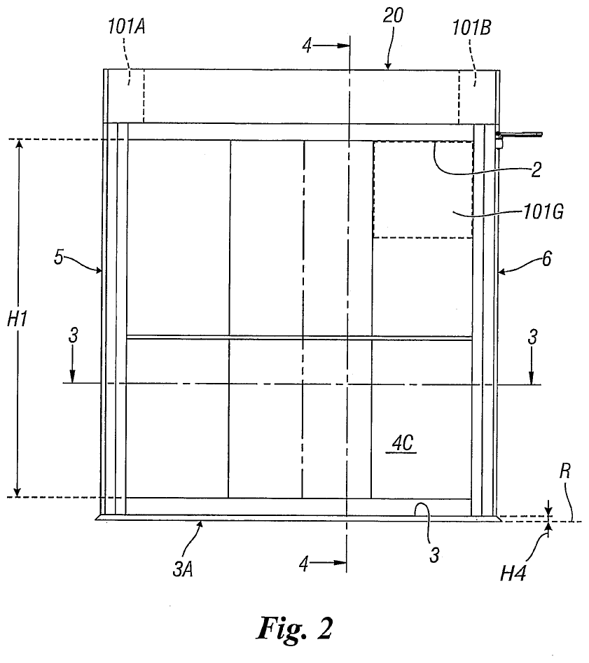

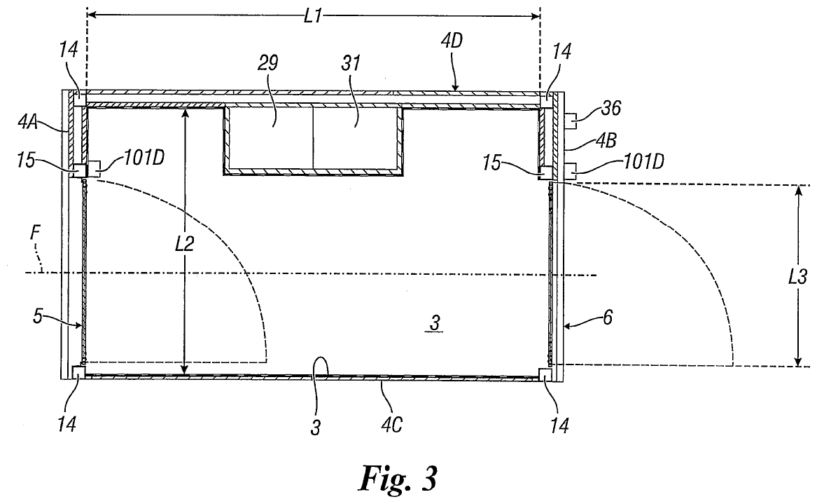

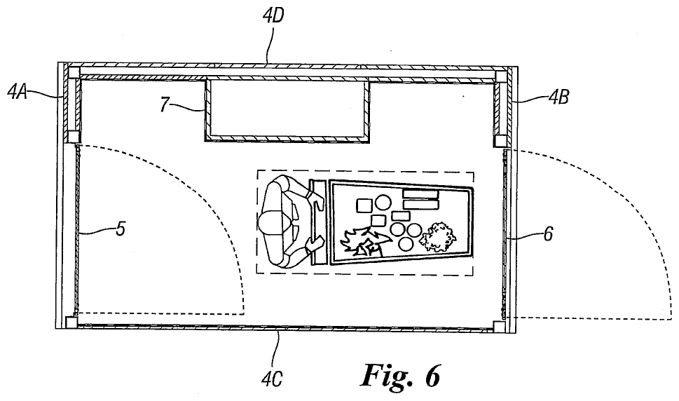

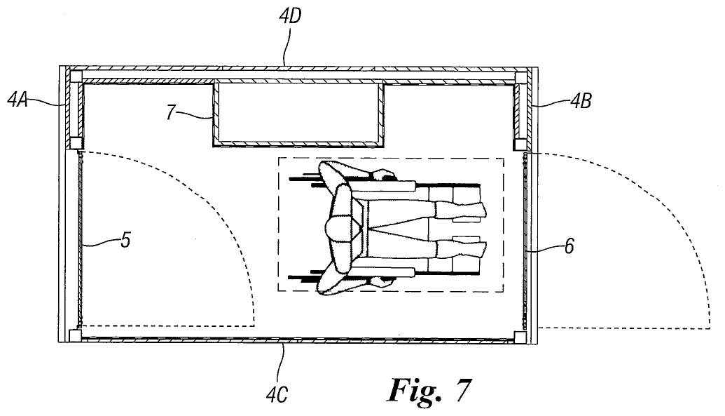

With reference to the structural characteristics of the cabin, it has preferably size such that it can house at least one person, or a person in a wheelchair or a person with a shopping trolley (as shown in FIGS. 6 and 7) inside it; to this purpose preferably the space L of the cabin 1 has the following dimensions:
length L1 (FIG. 3) at least 2000 mm, preferably between 2000 mm and 2500 mm, more preferably equal to about 2210 mm internal height H1 (FIG. 2) at least 2100 mm, preferably between 2100 and 2300 mm, more preferably equal to about 2200 mm,
internal width L2 (FIG. 3) at least 1000 mm, preferably between 1100 and 1300 mm, more preferably equal to about 1220 mm,
width L3 of the two doors 5 and 6 (FIG. 3) at least 800 mm, preferably between 800 and 900 mm, more preferably equal to about 850 mm.

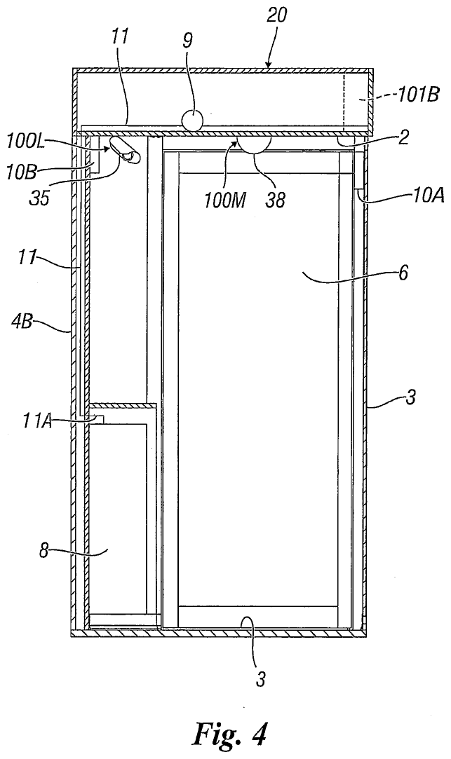

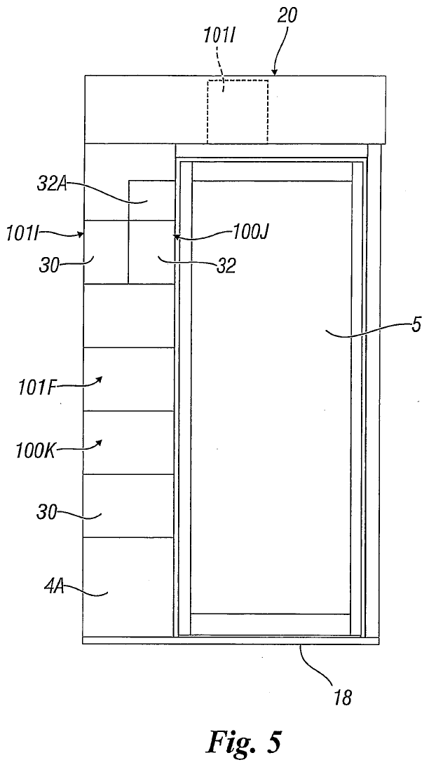

Preferably, the cabin has a very simplified support structure comprising a known rectangular base 3A (FIG. 2) which also defines the base wall 3 of the cabin, to which an upright 14 is rigidly constrained (FIG. 3) in correspondence with each of its four corners, whose upper end is rigidly constrained to the upper wall 2 of the cabin. In turn, in a known way the side walls 4A-C are fastened to and doors 5 and 6 are hinged to the uprights 14. The side walls 4A-D are walls of the known type having for example a thickness comprised between 1 mm and 10 mm. Advantageously, at least one of the side walls (in the shown example the wall 4C) is made of a transparent material, so as to make what happens in the cabin visible from the outside, and to be able to intervene, for example, in case the person inside performs undesired behaviors. Advantageously, at least one side wall 4D, 4A, 4B is made by means of two walls opposite and slightly spaced from each other (for example, by a distance between 10 mm and 100 mm) so as to create an hollow space for the passage of cables and/or the circuit for conveying the sanitizing substance to the members 10 emitting the sanitizing substance (as partially represented in FIG. 4).

The doors 5 are 6 are also of a conventional type for one skilled in the art; advantageously, to facilitate access to people in wheelchair and/or with a shopping trolley, the doors are arranged in opposite positions, that is with a longitudinal symmetry axis F (FIG. 3) in common, so as to create a continuous rectilinear passage for the wheelchair and the trolley. Advantageously the doors are hinged to the two angular uprights 14 that also support one of the greater side walls 4C of the cabin and come into contact with dedicated uprights 15 to which also the end edges of the smaller side walls 4A and 4B are hinged.

Advantageously, to facilitate access and exit from the cabin, the entrance door 5 opens towards the inside of the cabin, while the exit door opens towards the outside of the cabin.

Advantageously, each door has a known automatic movement element 101A 101B (partially represented in FIG. 2) for automatically moving them, and a device 101C 101D (partially represented in FIG. 3) for automatically locking and/or unlocking the two doors.

These elements 101A-D are of the known type and will not therefore be described in detail below; the automatic moving element of the door comprises for example an electric motor and transmission parts for transmitting motion from the motor to the door, while the locking/unlocking member comprises for example a known solenoid-type electric lock for interacting with a suitably shaped seat of the supports elements for the doors. Advantageously, also the doors comprise at least one part thereof which is made of a transparent material.

Advantageously, in correspondence with the two entrance and exit doors, the cabin has an inclined threshold 18 adapted to facilitate the access of the wheelchairs and trolleys, in any case advantageously the height H4 (FIG. 2) of the internal wall 3 of the cabin with respect to the external support surface R of the cabin is extremely moderate, for example between 5 mm and 50 mm, more preferably between 10 mm and 30 mm.

Advantageously, the cabin can have a floor, for example a self-disinfecting, or self-sanitizing and antibacterial PVC floor, of the conventional type for the one skilled in the art.

The cabin has an additional element 20, in correspondence with its upper wall 2, which forms the roof of the cabin, preferably having the same side sizes than the cabin itself and which is adapted to house, for example, the movement elements 101A 101B of the doors 5 and 6 and/or further components of the device which will be discussed below, and preferably also the controller 12.

Again, it is stated that the embodiment of the cabin has been provided only by way of example and that it could also include a different shape, for example a cylindrical shape, and for example even with a single tubular side wall.

For example, the cabin could also be of the “cylindrical” type usually used for access to banks.

According to a further variant, the walls of the cabin, regardless of the shape it has, could be made of a shatterproof or “armored” or bulletproof material of the same type used for the external walls or windows of banks and jewelers.

In a further, not shown variant, the cabin could also only have a single entrance and exit door.

According to further variants, the cabin could also have sizes different from the ones indicated, if it is used for specific uses; the cabin, for example, can be longer and larger (for example with sizes increased from 50% to 200% with respect to those previously indicated) to house several people at the same time: such as, for example, accompanied minors or accompanied disabled people, or a plurality of children for example of a school.

The cabin could also have even greater sizes if it is used with the permanent presence of specialized personnel inside it, for example if the cabin is used as a fixed position for a staff of doctors/nurses who operate on a high flow of patients, for example to carry out vaccinations, samplings, swabs.

The system according to the invention could also provide a plurality of identical cabins, instead of a single cabin, so as to provide some common walls and/or some components of the common sanitizing means (for example the sanitizing substance tank or the substance feeding member).

With regard to the sanitizing unit 101E (schematically shown in FIG. 8), the tank 8 of the sanitizing substance is of a conventional type and, preferably, as discussed below, it is housed inside the cabin in a dedicated housing member 31. The circuit 11 for conveying the substance from the tank 8 to a plurality of emission members 10, is also of the conventional type and provides known tubular elements 11A in plastic material provided with known connecting elements (not shown) for connection with the tank 8 and with the conveying member 9 and the emission members 10. As schematically represented in FIG. 4, the tubular elements advantageously extend in the hollow space of the side wall 4B and in the roof 20. The conveying member 9 comprises a known pump 9A adapted to convey the substance from the tank to the emission members 10 at a predetermined pressure, and preferably also a known compressor 9B adapted to suck air from outside the cabin and to convey it together with the substance to the emission members 10 so as to be able to atomize the substance in the cabin through said members 10. The emission members 10 comprise known nozzles 10A, B (FIG. 4) adapted to spray a substance, for example the nozzles are constrained to the upper wall 2 of the cabin, in correspondence with the two side walls 3 and 4C, and in the center of the cabin. Advantageously, the nozzles are inclined by about 45° towards the floor 3 of the cabin. Advantageously, the nozzles provide an inlet for the sanitizing substance which is liquid, and one for the pressurized air, which are mixed before being released. The nozzles are adapted to release the sanitizing substance in micro drops having dimensions between 2 and 7 microns, more preferably equal to about 5 microns, which create a sort of mist in the cabin which is however not perceived as wet given the size of the micro drops it is composed of.

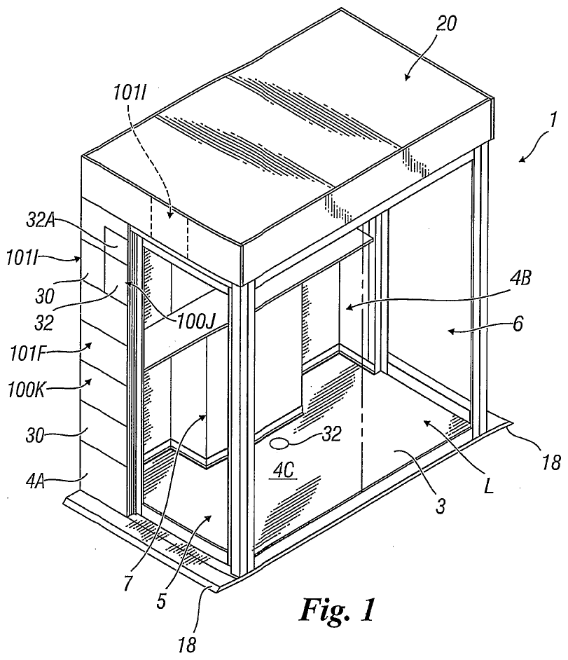

The sanitizing substance is preferably a known liquid substance based on hypochlorous acid, preferably of natural origin, more preferably 100% mineral, with neutral PH. To activate sanitation, the controller 12 receives an activation signal from at least a sanitation activation element 100E, the latter is of the type comprising:
door closure detecting members,
and/or members 100F for detecting the presence of a person in the cabin (for example a passage sensor adapted to detect a person entering the cabin)
   or a known activating member 32 (FIG. 1), for example of the button type, adapted to be activated by the person present in the cabin, for example by means of a foot, and to generate an input start signal Sa (FIG. 9) for the controller 12.

The controller 12 receives and processes the signal corresponding to the closing of the doors, and/or the start signal or the signal indicating presence of a person in the cabin, through the controller components 112 and 113, and once it has been verified that the doors are closed and that in the cabin there is a person who has activated the activation member a control signal is generated at the output for the conveying member 9A, 9B, so as to supply the emission members 10 with the sanitizing substance for a predetermined time.

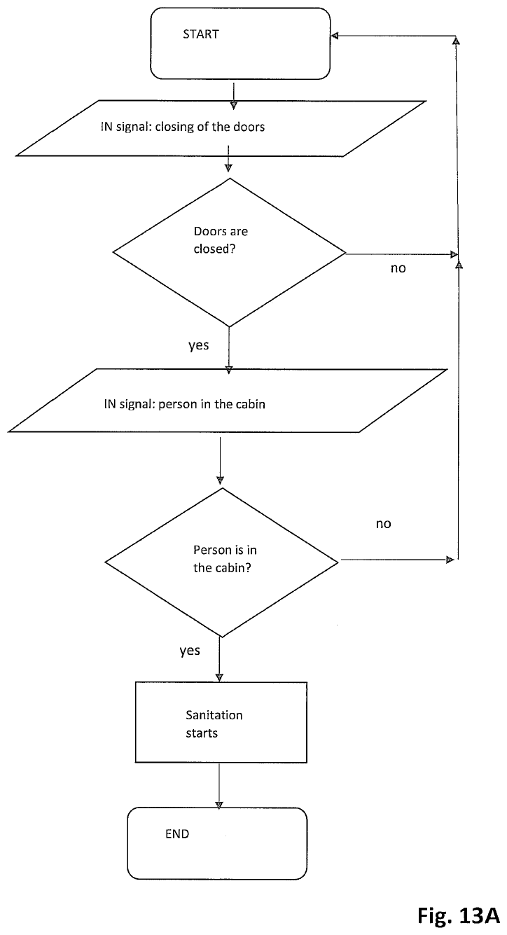

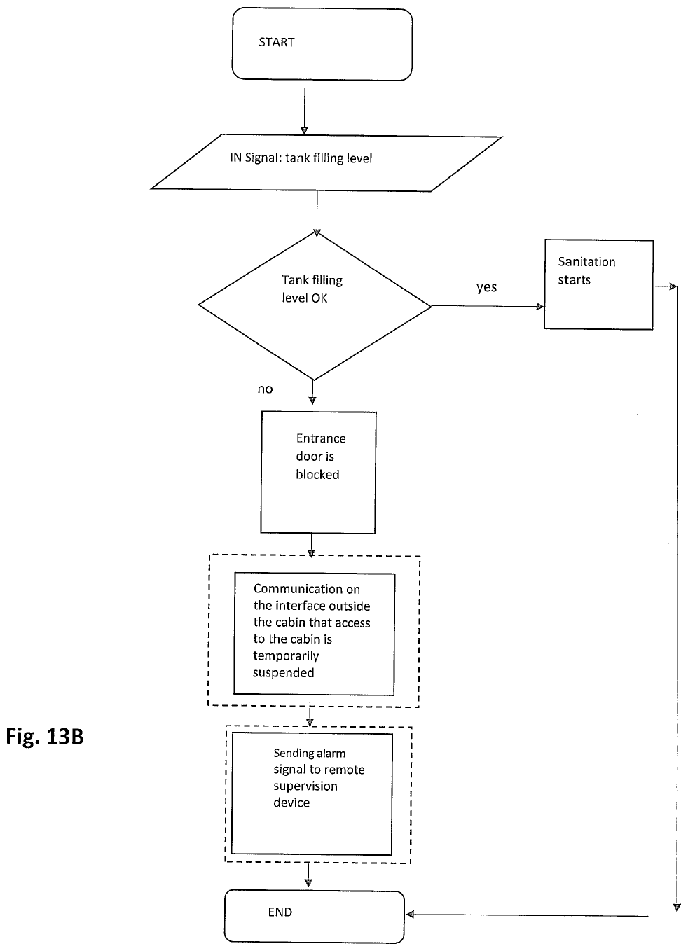

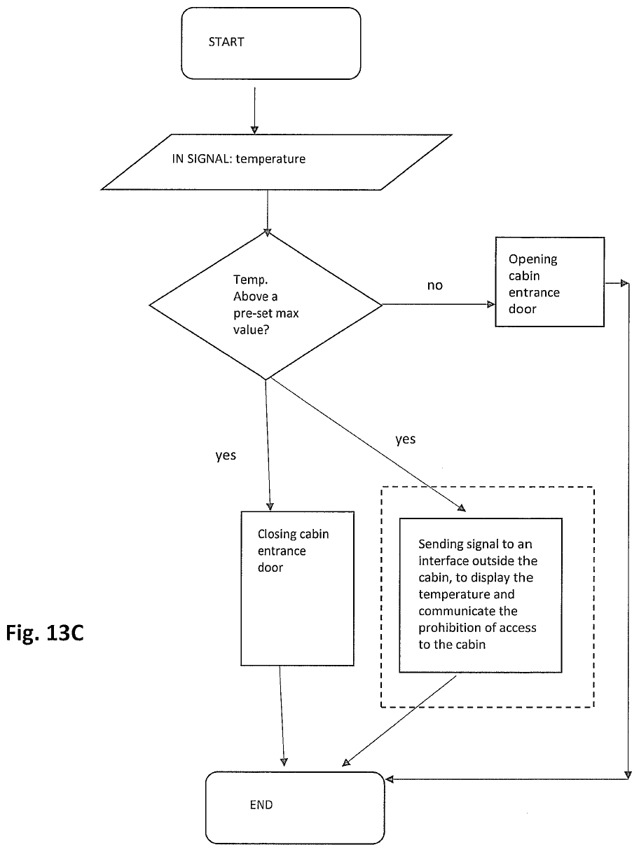

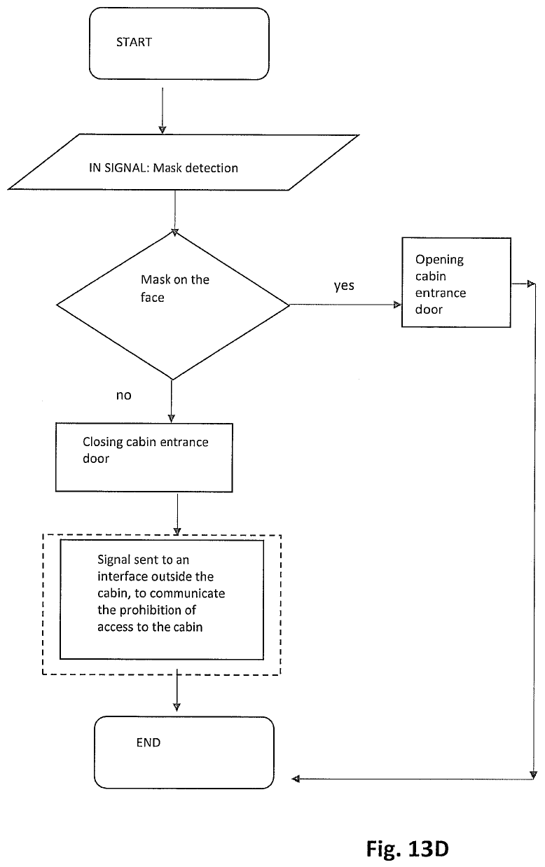

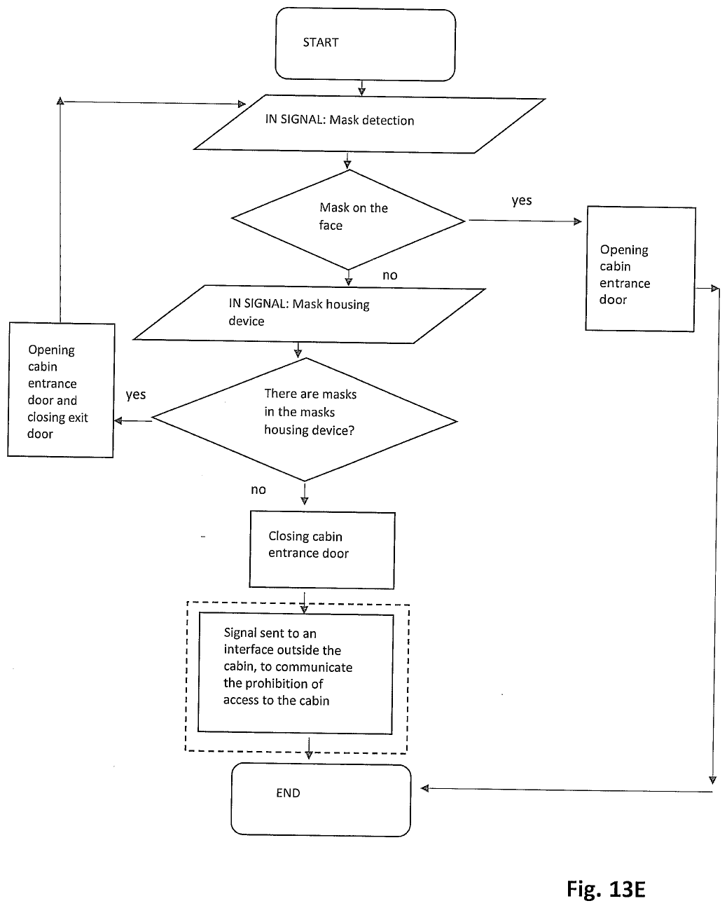

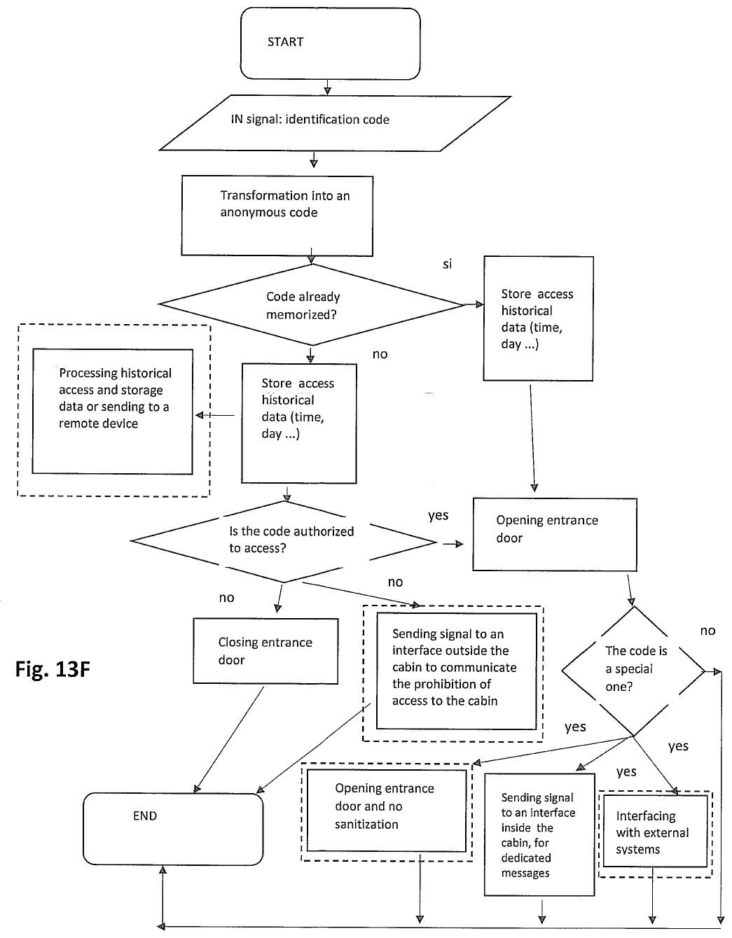

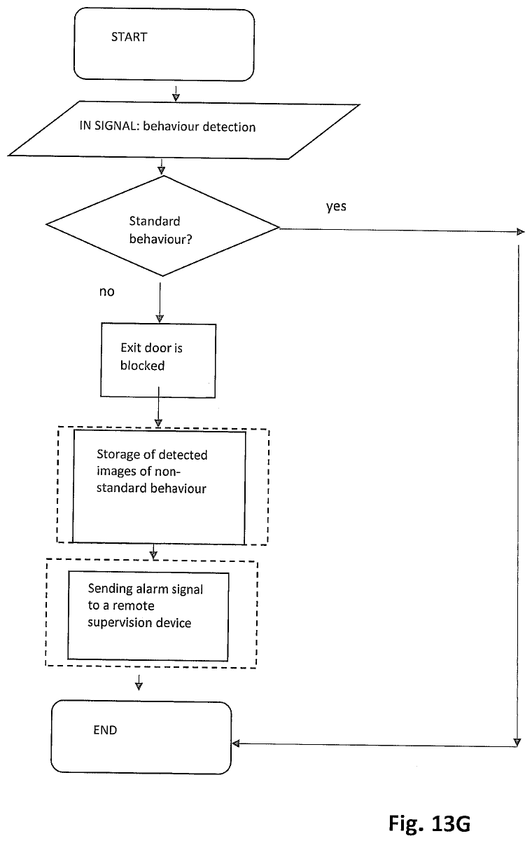

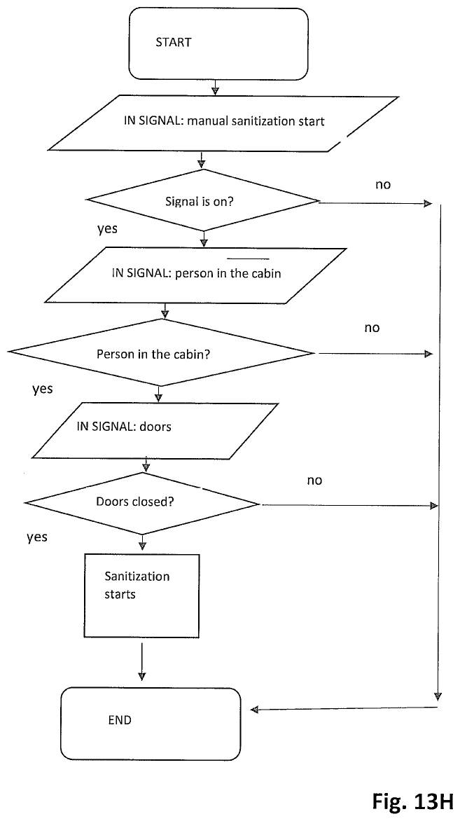

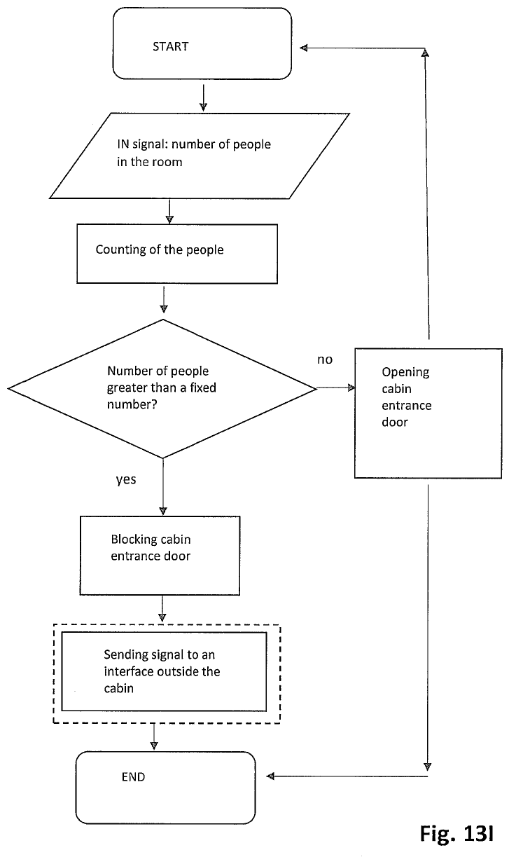

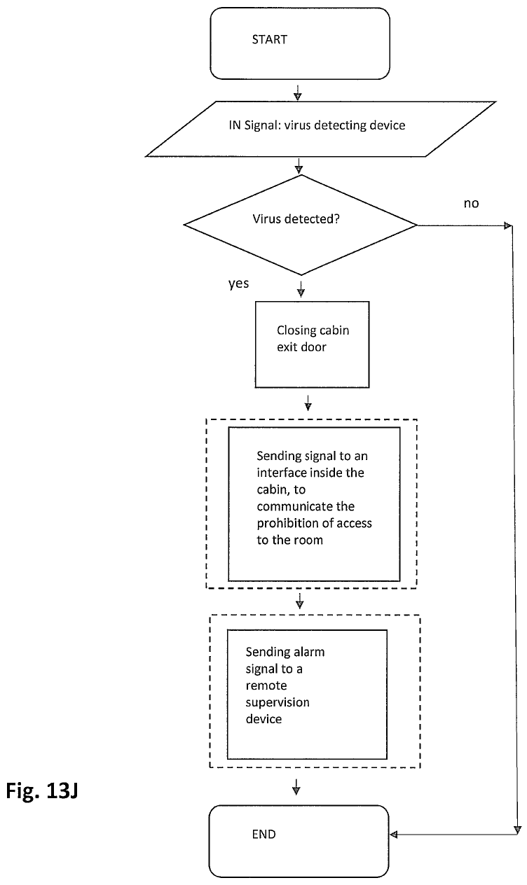

This sanitation activation process is illustrated in the algorithm of FIG. 13A.

The sanitizing unit 101E (schematically shown in FIG. 8) advantageously also provides other control functions; the control unit 12 in fact also controls the level of the sanitizing substance present in the tank 8, for this purpose, for example, detection devices 100D are provided, for example a load cell, adapted to detect the tank filling state. These detection devices 100D are connected to the controller 12 and generate a monitoring signal relating to the tank filling level for the controller components 112, 113 (FIG. 9) for the reception and processing of said level signal, adapted to evaluate the tank filling level, and to generate a second control signal related to said evaluation which is adapted, for example:
to generate on an interface 23 (FIG. 1) provided outside the cabin at the entrance door 5, for example a known screen, an information (for example of a visual type) adapted to inform people who want to access to the cabin that the cabin is no longer able to perform sanitization,
and/or is adapted to lock the entrance door 5, (acting on the locking device 101C) until the tank is replaced with a full one.

This sanitation activation process is shown in the algorithm of FIG. 13 B (wherein the optional or alternative functions discussed above have been represented within a dashed frame).

Advantageously, the control unit 12 sends an alarm message (“tank exhausted” or “tanks nearing exhaustion”), via the controller component 114, for example by means of a network Ri (schematically shown in FIG. 9A), to a remote supervision device 200.

The sanitizing unit 101E (schematically shown in FIG. 8) advantageously also provide other control functions: the control unit 12 also controls:
measuring unit 100N for measuring the pressure in the circuit supplying the substance and/or the air to the emission organs 10, adapted to detect for example if said circuits are under pressure or a pressure change has occurred,
a detecting element 1000 for detecting the malfunction, for example of the blockage of the emission members 10,
a detecting element 100P and 100Q for detecting a breakdown in the compressor 9B or in the pump 9A,
detecting elements 100P and 100Q for detecting a breakdown in the compressor.

As illustrated with reference to the detecting element 100D for detecting the tank 8 level, the command and control unit 12 also receives the signals of these further detecting elements 100N-P and is adapted to generate corresponding control signals related to the evaluation of the monitoring signals received at the entrance, for example for the interface 23 and/or for the locking device 101A, 101C of the entrance door 5, and/or for sending an alarm signal to a remote supervision device 24. According to a variant shown in FIG. 9B, the device according to the invention could comprise a controller 12′, of the type substantially identical to the one 12 described so far, but which is specific and only dedicated to the sanitizing unit (the components of this controller 12′ will not be described in detail below and will be indicated with the same numbering used for the controller 12 with the addition, however, of a superscript).

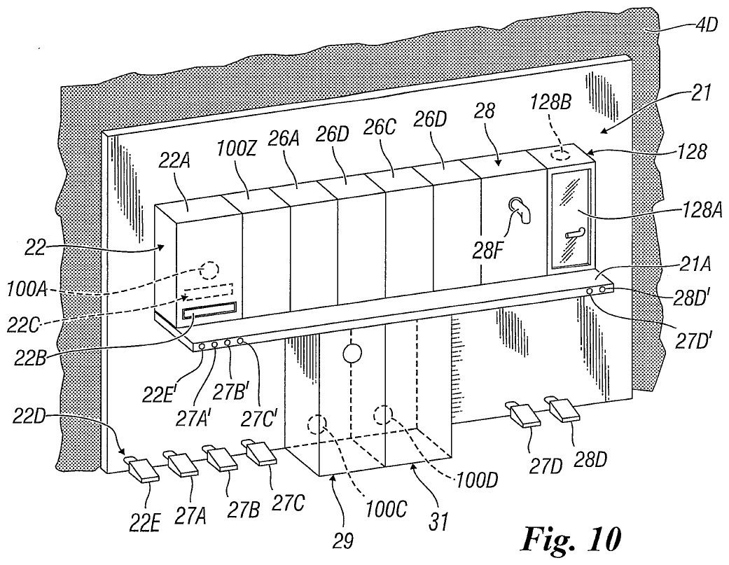

Inside the cabin, according to a preferred embodiment, preferably in correspondence with the wall 4D opposite to the one 4C the doors 5 and 6 are hinged to, there is an equipped structure 21 (schematically shown in FIG. 10) comprising at least one housing device 22 adapted to house, for example stacked one on top of the other, a plurality of known protection masks of the type used to protect against viral infections, for example from covid-19. The housing device 22 for example comprises a box-shaped container 22A adapted to contain the masks, and provided with a dispensing slot 22B for dispensing masks and with known elements 22C (for example of the type having a motorized pusher element) provided in the container 22A, for automatically dispensing a mask at one time, when operated by a person in the cabin, a control element 22D preferably of the type comprising a pedal 22E, so as to be able to control the elements 22C even by means of the pressure applied by a foot. Advantageously, the element 22 also comprises a detecting element 100A adapted to detect if there are still some masks in the container 22A and advantageously to also provide the number of masks still present in this container, said element 100A comprises for example a known load cell adapted to weight the masks present in the container 22A, alternatively they could also comprise a simple level sensor.

The mask detecting element 100A is connected to the controller 12 and generate a first “mask presence” monitoring signal for the reception and processing controller components 112, 113 (FIG. 9), adapted to assess whether masks are still present in the housing device 22 for housing the masks, and to generate a control signal related to said assessment which is for example sent by the controller 12 to an interface 101F (FIG. 1) provided outside the cabin in the correspondence with the entrance door 5, for example a known screen, adapted to display a predetermined information on said interface (for example of the visual type) adapted to inform people who want to access to the cabin that the cabin has run out of masks. Advantageously, the controller 12 also provides a controller component 114 for sending alarm messages (“lack of masks”), to the remote supervision device 200. Preferably, the equipped structure 21 also has a shelf 21A arranged below the mask housing device 22 and adapted to facilitate the mask withdrawal.

The equipped structure 21 can also optionally have other elements 26A-D (FIG. 10) for supplying protection devices (for example protection gloves, footwear, headgears, gowns, glasses) which will not be described in detail below as they are substantially identical to the mask housing device 22, and each one provided for example with control elements comprising pedals 27 A-D and with elements adapted to automatically detect is the supplying device still contains the protection devices they should supply, and/or the amount of protection devices still contained in said supplying device.

The equipped structure 21 can also optionally have a device 28 (FIG. 10) for dispensing a hand sanitizing substance which, preferably, is contained in single-dose sealed packages; this device 28 will not be described in detail below as it is substantially identical to the mask housing device 22, said device 28 for example comprising a control element having pedals 28D and a sensor adapted to automatically detect whether the dispensing device still contains the gel packages they should dispense, and/or the amount of packages still contained in said dispensing device. The device 28 for dispensing a hand sanitizing substance could also be of the type adapted to dispense dosed quantities of a liquid substance contained in a bottle, in which case it would be provided with a dispensing spout 28F and with a dispensing pump operated for example by the pedal 28D. This variant, too, would be preferably equipped with a sensor adapted to automatically detect if the device still has sufficient substance to dispense, and/or the quantity of substance still contained in the bottle provided in said dispensing device. Advantageously, a cabinet 128 (FIG. 10) having an openable door 128A is also provided in the cabin for sanitizing particular objects, such as for example mobile phones, laptops, tablets, and so on. This cabinet has internal sanitation devices 128B, for example of the type comprising known UCV led. This cabinet is for example arranged side by side with the supplying devices 22A, 26A-D, 28 previously described (FIG. 10).

The equipped structure 21 can also optionally have, for example under the shelf 21A a basket 29, for example to throw away the packages of the masks or of other protection devices or of the sanitizing substance (if contained in single-dose packages); this basket has preferably a detection sensor 100C, for example a load cell, adapted to detect the basket filling state. This sensor 100C is connected to the controller 12 and generate a monitoring signal relating to the filling of the basket for the controller components 112, 113 (FIG. 9) for receiving and processing said filling quantity, adapted to evaluate the basket filling state, and to generate a control signal related to said evaluation that is sent by the controller 12 to an interface 101B (FIG. 1) provided outside the cabin at the entrance door 5, for example a known screen, adapted to display on this interface a predetermined information (for example of the visual type) adapted to inform people who want to access to the cabin that the cabin is no longer able to receive wastes in its basket. Advantageously, the controller 12 also sends an alarm message (“basket full”) through the controller component 114, to the remote supervision device 200.

In order to allow the aforementioned various devices to be operated even by people in wheelchairs, it is desired to emphasise that all the pedal commands 27A-D and 28D described so far are advantageously also complemented by known buttons provided in the cabin in positions that are easily accessible to people in a wheelchair; for example the can be arranged for example along the edge of the shelf 21A, as shown in FIG. 10, where these additional commands have been indicated with the same numbering used for the pedal commands with the addition, however, of a superscript.

The system according to the invention advantageously also provides a unit 100I for automatically measuring the temperature of the person who wants to enter the cabin, which is connected to the controller 12 and adapted to generate a first monitoring signal relating to the measured temperature. According to the invention, the receiving 112 and the processing 113 controller components of the controller 12 are adapted to process the temperature monitoring signal and check whether the measured body temperature is higher than a predetermined limit value, and to generate a first control signal related to said check, for the device 101C (partially shown in FIG. 3) for automatically locking/unlocking the electric lock of the cabin entrance door 5, and/or for the movement device 101A (FIG. 2) for automatically moving the entrance door 5. These processes related to the temperature measurement are shown in the algorithm of FIG. 13C (wherein the optional or alternative functions discussed above have been shown with a dashed frame).

The unit 101I for automatically measuring the body temperature of the person who wants to enter the cabin is of a conventional type for the one skilled in the art, and comprises for example a known thermal scanner adapted to immediately measure the body temperature of the person present in front of the thermal scanner itself and to send a monitoring signal relating to the measured temperature to the controller 12.

If temperature is desired to be measured more accurately, the temperature measuring unit 101I comprises one or more known thermal sensors 30 (FIG. 1) placed at different heights, and a known video camera 32. Advantageously, two thermal sensors 30 arranged at different heights (for example, one between 0.8 m and 1.30 m and one between 1.30 m and 1.90 m), a video camera 32 and a proximity sensor 32A are provided, in this way the unit 101I allows to measure the precise temperature of the person, whether he/she is an able-bodied or a disabled in a wheelchair or parent and son/daughter at the same time at different heights; the proximity sensor allows the thermal sensors to calibrate the distance for recognition and for activating a precise measurement. The controller processes the signals received from the sensors and the camera in a conventional way for the one skilled in the art, for example using artificial intelligence techniques and one or more neural networks, as described for example in the Italian patent application No. IT201800009442A in the name of the company Laser Navigation incorporated by reference in the present text. The controller 12 and in particular the processing controller component 113 therefore comprises a further recognition controller component 115, for example artificial intelligence algorithms, adapted to recognize the person's face on the basis of the information received from the camera and to only identify and select the information on the temperature measured by the sensors in correspondence with the sensors (preferably in one or more precise points of the face, for example at the person's forehead) and not with other parts of the body, so as to get a temperature measurement accuracy of the order of +/−0.3° C. More specifically, the processing controller component 113 recognizes the face “object” and the neural network recognizes whether what is head-on on the camera is a face, when it corresponds to a series of points of the camera tracing, and calibrates the correct distance through the proximity sensor to obtain a temperature measurement with a deviation of +−0.02° C.

Advantageously, the receiving 112 and the controller component 11 of the controller 12 also generate a second control signal related to the temperature verification for the interface 101F (FIG. 1) provided outside the cabin at the entrance door 5, for example a known screen or a known loudspeaker, adapted to display or communicate through said interface the measured temperature value, and if it exceeds a predetermined limit, to communicate a predetermined message informing the person that he/she is not allowed to enter the cabin because the measured temperature is higher than the predetermined one.

The device according to the invention advantageously also provides a unit 100J for automatically detecting the mask on the face of the person who wants to enter the cabin, which is connected to the controller 12 and adapted to generate a first monitoring signal relating to the mask. According to the invention, the receiving 112 and the processing 113 controller components of the controller 12 are adapted to process the mask monitoring signal and to verify whether or not the person who wants to enter wears a mask on his/her face, and to generate a first signal related to this verification. According to a first variant of the invention, the control signal relating to the mask verification controls a device 101A 101C for locking the opening of the cabin entrance door 5 (for example an electric lock or a device for the door automatic movement), so as to lock the opening of the entrance door if a mask has not been detected on the face of the person who wants to enter the cabin, and preferably to report the denial of access even through an interface placed outside the cabin. These processes related to the mask control are shown in the algorithm of FIG. 13D (wherein the optional or alternative functions discussed above have been represented within a dashed frame).

According to a second variant of the invention, the cabin has an internal mask housing device 22 for housing and supplying masks and the control signal relating to the mask control operates a device 101B, 101D for locking the opening of the cabin exit door 6, so as to lock the opening of the exit door if a mask has not been detected on the face of the person who wants to enter the cabin, so that said person without the mask can withdraw a mask from said mask housing device 22 and can then get out of the cabin from the entrance door to perform the check again on the presence of the mask of the face.

Advantageously, this second variant of the invention is operated only if the control unit 12 preliminarily verifies, thanks to the detecting element 100A (adapted to detect if there still are masks in the container 22A of the mask housing device) that the mask housing device 22 still has masks to supply. If this preliminary check is negative, the person is not allowed inside the cabin.

This process is shown in the algorithm of FIG. 13E (wherein the optional or alternative functions discussed above have been represented within a dashed frame). The unit 100J for automatically detecting the mask on the person's face comprises for example one or more known video cameras and one or more proximity sensors, for example the same video camera 32 and a proximity sensor 32A used by the temperature measurement devices 100I. The image or images provided by the video camera and the proximity sensor signals are analysed by the controller 12 and in particular by the controller component 113 which, for this purpose, comprises for example a further facial recognition controller component that is advantageously the same facial recognition controller component 115 mentioned for the facial recognition related to the body temperature measurement. Thus, also in this case, preferably the unit 100J for automatically detecting the mask are artificial intelligence algorithms.

The facial recognition techniques and systems that can be used are very numerous, for example it is possible to use the already severally mentioned system that analyzes the image through artificial intelligence techniques and that uses neural networks of the type described in the Italian patent application No. IT201800009442A in the name of the Italian company Laser Navigation, incorporated in the present text by reference.

The system according to the invention advantageously also provides a unit 100K for identifying the person who wants to enter the cabin, which is connected to the controller 12, and is adapted to generate a monitoring signal relating to the identification. According to the invention, the receiving 112 and processing 113 controller components of the controller 12 are adapted to process the identification monitoring signal and activate a plurality of control members, which are described below in detail. According to the invention, the identification unit comprise any known device adapted to receive a code or an information which allows to identify a person; the devices of this type are innumerable and depend upon the way in which the person's identity is coded and/or upon the technology through which said identity is communicated to the identification device.

By way of example, the identification device comprises:
known magnetic stripe card readers (for example loyalty cards of a shop, electronic identity cards, health cards, ATMs, and so on)
known bar code or Qr code readers,
known RFID tag readers,
known Bluetooth signal receivers,
a known video or photo camera,
a keyboard or a microphone adapted to detect an acoustic message,
known biometric sensors such as, for example, recognition of the retina and/or iris, fingerprints.

It should be noted that the detected identification code can be of the absolutely anonymous type, for example a numeric code, or it may contain sensitive information for the person's privacy, the person's face or the data reported on his/her electronic identity card.

Preferably, the detected identification code as well as the camera's video stream and the temperature measurement are immediately transformed into metadata at the time of detection, so that the sensitive stream data are not stored in the controller 12 which, therefore, preferably neither processes nor store these sensitive data.

In case identification occurs through an image obtained by means of a video or a photo camera, for example the same video camera 32 used for the temperature measurement devices 100I, the image or images provided by the video/photo camera are analyzed by the controller 12 and in particular by the processing controller component 113 which, for this purpose, comprise for example a further facial recognition controller component which advantageously is the same facial recognition controller component 115 mentioned for the facial recognition related to the body temperature measurement. Thus, in this case as well, preferably the unit 100K for identifying the person who wants to access the cabin are artificial intelligence algorithms.

The controller 12 carries out a plurality of processes on the identification code received.

First of all, it verifies whether this code is already present in a controller component or data base 116 of the controller 12 (for example in the form of metadata, or an anonymous numeric code, or an already previously stored face's image; if not present, it stores the code (or the correspondent metadata) and the temporal access data, while if already present, it adds the temporal data of the required access to the already present data.

The controller 12 through the processing controller component 113 verifies, by means of the data base 116 if the identification code corresponds to a person who has been allowed or denied permission to access (for example because considered undesired) and, in case access has been denied, automatically blocks the entrance door 5 by generating a control signal for the door opening locking devices 101A or 101C previously described and a control signal for the interface 101F (FIG. 1) provided outside the cabin at the entrance door 5, for example a known screen, adapted to display a predetermined information on said interface (for example of the visual type) adapted to inform the person who wants to enter that he/she has been denied permission to access, preferably also indicating the reason for this decision.

The controller 12 through the processing controller component 113 is also able to process a historical analysis of the data of the person accessing the cabin, for example the data on the greatest flow time period and duration of the use of the cabin. These processes are advantageously stored by the controller 12 for example in a storage controller component 117 and/or sent to the remote supervision device 200 previously described.

Through the processing controller component 113 the command and control unit 12 is also able to identify whether particular operations of the cabin are foreseen for a specific identification code, for example to pass through the cabin without activating the sanitization (for example in situations of emergency) or to generate a customized control signal for the interface 101F provided outside the cabin or for an additional interface 101G (FIG. 3) provided inside the cabin, the latter comprising for example a screen and a loudspeaker or, more advantageously a smart TV. In particular, the cabin control unit 12 is adapted to generate control signals for these interfaces so that they communicate customized messages to the person.

In situations of emergency the cabin advantageously provides known devices, not shown, to unlock the entrance which are operated by the service staff (security/internal operators or other) who can facilitate the doors unlocking by means of different types of identification. This identification processes are shown in the algorithm of FIG. 13F (wherein the optional or alternative functions discussed above have been represented within a dashed frame).

The cabin also has a known autonomous electrical continuity group so as not to undergo alterations caused by any temporary electrical blackouts.

The controller 12 can interface with external systems such as a known access control system, and/or an automatic operator, and/or external video surveillance systems to which it communicates the results of the identification code detection and/or the temperature measurement and/or the safety systems of the place a person wants to access.

If, for example, the system is provided for access to commercial premises, such as for example a supermarket, the controller 12, on the basis of the identification code can also activate for example on an interface 101G provided inside the cabin, described in detail below: promotional messages on the bases of the previously stored tastes of the identified person, and/or it can recall any on-line, active orders previously stored, and direct the customer to the casher for collection.

The controller 12, if for example the system is provided for access to a place open to the public, such as for example service centers, offices, clinics, on the basis of the detected identification code it can activate, for example on an interface 101G provided inside the cabin, described below in detail: messages for directing the identified person to the booked office, on the basis of the previously stored data of the identified person, or it can inform the recipient of the meeting with the identified person for a more effective and customized welcome, so as not to have to go through the reception/secretary, and thus to limit the possibility of any contagion. In the latter case, the controller 12, as already indicated above, provides a known interface controller component for interfacing with external systems such as a known access control system, and/or an automatic operator, and/or external video surveillance systems to which it communicates the results of the identification code detection and/or the temperature measurement.

As already indicated above, advantageously the system has an interface device 101G (FIG. 3) inside the cabin for example constrained to the wall 4D which also has the equipped structure 21, said interface device comprises for example a screen and a loudspeaker or, more advantageously a smart TV. In particular, the controller 12 is adapted to generate control signals for the interface 101G so that it communicates predetermined messages to the person, which are stored in a controller component of the controller 12 for example in a memory 118.

These messages can for example provide an explanation on how to correctly wear the mask and/or other protection devices, on how to start sanitation, on the duration thereof and on how sanitation will occur, on whether or not it is necessary to withdraw a mask and to have to carry out the test again for verifying that the mask has been worn However, the interface 101G can advantageously also display, for example during the sanitation procedure, casual or predetermined messages of the promotional type and selected according to the person's identification code, as discussed above.

Advantageously, the system also has a detecting unit 100L (FIG. 4) inside the cabin for detecting the performed actions, that is the person's behavior into the cabin, which is connected to the controller 12 and is adapted to generate a first behavior monitoring signal. According to the invention, the receiving 112 and processing 113 controller components of the controller 12 are adapted to process the behavior monitoring signal and verify whether the person is behaving in a “non-standard” or “not predetermined” way, and to generate a first signal related to said verified behavior.

The “non-standard” behavior can be manifold; they comprise, for example, that of a person leaving the cabin without undergoing the sanitation process, or a person withdrawing more than one mask.

The detecting unit 100L comprises for example a known video camera for example constrained to the cabin upper wall 2 as shown in FIG. 4. The image or images provided by the video camera are analyzed by the controller 12 and in particular by the controller component 113 that, for this purpose, comprise a further dedicated controller component 119 which preferably is able to recognize the particular “non-standard” behaviors through a neural networks and artificial intelligence systems. The “non-standard” behaviors recognition techniques and systems that can be used are manifold; for example it is possible to use the already severally mentioned system which analyzes the image through artificial intelligence techniques and uses neural networks of the type described in the Italian patent application No. IT201800009442A in the name of the Italian company Laser Navigation, incorporated in the present text by reference.

Further to the detection of a “non-standard” behavior, the controller 12 generates a first control signal for the device 101B, 101D for locking the opening of the cabin entrance door 6, so as to lock the opening of the exit door.

Alternatively or in addition to the above, the control unit 12, further to the identification of a “non-standard” behavior, can send an alarm message via the controller component 114 (“abnormal behavior of the person in the cabin”), to the remote supervision device 200 and to proceed with the storage, for example in a controller component 117, of the images taken by the video camera 35. These processes related to the “non-standard” behaviors are shown in the algorithm of di FIG. 13G (wherein the optional or alternative functions discussed above have been represented within a dashed frame).

Advantageously the sanitation process can be implemented even if there are no people in the cabin. In fact, the cabin could be desired to be sanitized before being used by people, or for example to sanitize objects that have been placed in the cabin, for example clothes that have been tried by a customer in a shop.

The device comprises members 36 (FIG. 3) for starting sanitation without the presence of people, provided for examples outside the cabin near the exit door 6, these members for example have a known conventional button or preferably a key switch or a code button so as to avoid unintended inputs. The controller 12 receives a signal from the member 36 and activates the sanitation unit 101E (as already previously shown with reference to the sanitation of person present in the cabin). Advantageously, the sanitation unit can have different sanitation times, for example longer times for this sanitation without the presence of people.

Advantageously the controller 12 also verifies that actually no people are present in the cabin before activating the sanitation unit; the presence of people is checked by verifying the monitoring signal received from a conventional presence 38 detecting element 100M (FIG. 4) for example of the infrared type, and only if the check is negative, that is no people have been detected into the cabin, it sends a control signal to the sanitation unit 1001E.

These processes related to the externally activated sanitation are shown in the algorithm of FIG. 13H (wherein the optional or alternative functions discussed above have been represented within a dashed frame).

The system also provides detecting device 200A-D, adapted to automatically and continuously detect people present in a room 40, in this context the term “room” means a place comprising one or more separate rooms, for example a supermarket, or an office.

According to the invention the system the detecting device 200A-D is adapted to generate a monitoring signal relating to people detected in the room, for the controller 12, of the cabin,
wherein the receiving 112 and processing 113 controller components of the controller 12 of the cabin 1 receive said people monitoring signal and verify whether the detected number of people is less or equal to a maximum number of people predetermined for said room, and generate a first control signal related to this verified number of people, for the device 101C 101A for automatically locking/unlocking the opening of the cabin entrance door 5, adapted to unlock said entrance door 5 if the number of people detected in the place is less than said maximum predetermined number and vice versa to lock said entrance door.

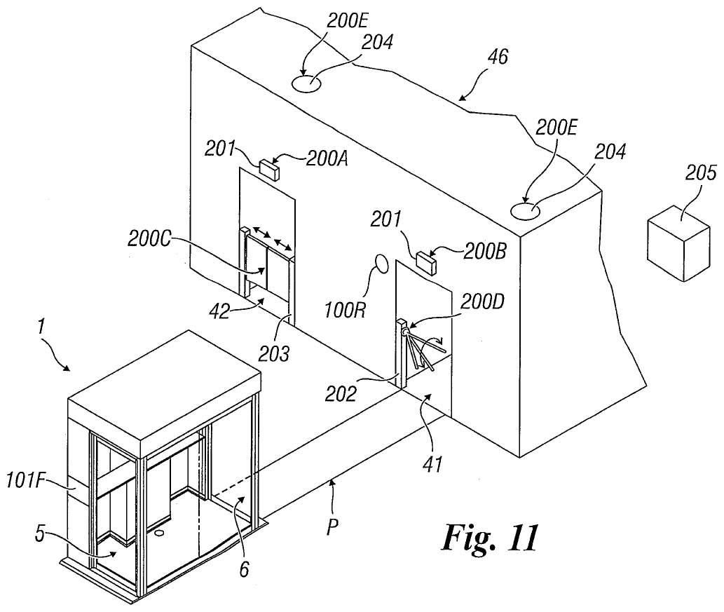

The cabin 11 as shown in FIG. 11 is preferably placed along an access passage P for the access opening 41 of the room 40, or according to a variant not shown it itself is a structure for direct access of people into the room 40, that is it is directly put close to the wall 40A of the room 40, at the access opening 41.

The detecting device 200 is of a conventional type for the one skilled in the art and can be of different types. They are adapted to automatically and continuously detect in the time the number of people present in the room, so as to provide the controller 12 with always updated data relating to the number of people actually present in the room 40.

They comprise, for example, a plurality of conventional electronic people-counting devices 201, for example of the infrared rays type or photocell type or thermal or optical camera, arranged in correspondence with the access opening 41 and the exit opening 42 of the room, in case this has separated entrance and exit doors. An alternative solution is for example to provide the entrance and exit doors each with a known device 202, 203 of the type adapted to move people one person at a time and in one predetermined direction only, for example devices 202 of the turnstile type (schematically shown in FIG. 11 in correspondence with the entrance 41), or devices 203 of the type having two movable bulkheads (schematically shown in FIG. 11 in correspondence with the exit opening 42); these devices are provided with known people-counting electronic sensors. In the event the place 40 has a single door acting as both an entrance door and an exit door to count people, it is always possible to use the aforementioned devices 202, 203 which, in this case, are of the type adapted to allow the passage of one person at a time, but in both directions, that is both entering and leaving, and provide known people-counting electronic sensors adapted to discriminate between people entering and leaving the place 3 and to provide the controller 12 with two corresponding monitoring signals: one for people leaving and one for people entering.

Alternatively, by providing the room 40 with a plurality of video cameras 204, which could be for example the known surveillance video cameras which are often present in the rooms, adapted to generate a plurality of signals for a processing unit 205 (FIG. 11) adapted to obtain the number of people present in the room from said signals, wherein said processing unit preferably calculates the number of people present in the room at the same time, through control systems that analyze the images generated by said video cameras and, calculates the required number of people for example, through artificial intelligence techniques and the use of neural network; a system adapted to carry out this function is for example the already severally mentioned system of the type described in the Italian patent application No. IT201800009442A in the name of the Italian company Laser Navigation.

Advantageously, the receiving 112 and processing 113 controller components of the controller 12 also generate a second control signal for the interface 101F (FIG. 1) provided outside the cabin at the entrance door 5, wherein said second signal is related to the verification of the number of people detected in the room and if this number equals or is greater than the maximum predetermined number of people, so as to communicate a predetermined message informing that the room to which one persons wants to access has reached the maximum number of people that it can receive and that it is necessary to wait before accessing said room again.

These processes related to verifying people present in a place where it is desired to access are shown in the algorithm of FIG. 13I (wherein the optional or alternative functions discussed above have been represented within a dashed frame).

Finally, it must be underlined that, according to the invention: the unit 100I for the temperature measurement, and/or the unit 100J for automatically detecting a mask presence/absence, and/or the unit 100K for identifying the person who wants to enter the cabin, and/or the detecting device 200A-D adapted to detect and/or count the people present in a room 40 where access is desired and to generate a monitoring signal relating to the number of people detected in the room, and/or the devices 101Y for controlling the sanitizing substance, which are described below in detail, and/or the devices 100Z for detecting the virus in a person, which are described below in detail can be provided even not in combination with a cabin 1 of the type described so far and/or any known cabin having known person sanitizing unit of other types.

Therefore, according to the invention, the unit 100I for the temperature measurement, and/or the unit 100J for automatically detecting a mask presence/absence, and/or the unit 100K for identifying the person who wants to enter the cabin, and/or the detecting devices 200A-D adapted to count the people present in a room 40 where access is desired, and/or the devices 101Y for controlling the sanitizing substance, which are described below in detail, and/or the devices 100Z for detecting the virus in a person, which are described below in detail, can be associated to a structure and/or an access element adapted to allow people to access a room, of the known type, in any case conformed, for example, to a known door, and/or to a known turnstile, and/or to sanitizing devices of the known type, and/or to a conventional a cabin or security portal for accessing a place requiring a security access, such as for example a bank or jewelry store.

The invention, therefore, also relates to a device for controlling people who want to access a room 40, adapted to at least reduce the risk that people can bring pathogenic contaminants into said room 40, in particular viral pathogenic contaminants, such as for example the coronavirus.

comprising:
an access element adapted to allow the entrance to said place,
a unit 100I for the measurement of the temperature of people who want to access said place,
and/or a unit 100J for automatically detecting the presence/absence of a mask on the face of people who want to access said place,
and/or an identification unit 100K of people who want to access said place,
and/or a detecting device 200A-D, adapted to count the people present in said room 40 to which access is desired, and to generate a monitoring signal relating to the number of people detected in the room,
and/or a device 101Y for controlling the sanitizing substance, described below in detail,
and/or a device 100Z for detecting the virus in a person, described below in detail,
at least one controller 12 of said unit 100I for the temperature measurement, and/or of the unit 100J for a mask presence/absence automatic detection, and/or of the unit 100K for identifying the person who wants to enter the cabin, and/or of the detecting device 200A-D, adapted to count the people present in a place 40 to which access is desired, and/or of the device 101Y for checking the sanitizing substance, described below in detail, and/or of the device 100Z for detecting the virus in a person, described below in detail;
wherein, preferably, the unit 100I for the temperature measurement has one or more of the features set forth in claims 2 and 5;
wherein, preferably, the unit 100J for a mask presence/absence automatic detection, has one or more of the features set forth in claim 3;
wherein, preferably, the unit 100K for identifying the people who want to access said place has one or more of the features set forth in claims 4, 18 and 19;
wherein, preferably, the detecting device 200A-D, adapted to count the people present in said place 40 have one or more of the features set forth in claim 11;
wherein, preferably, the devices 100Z for detecting the virus in a person, described below in detail, have one or more of the features set forth in claim 14.

Finally, it must be underlined that the embodiments shown so far have been provided only by way of example and that numerous variants are possible, all falling within the same innovative concept, thus for example the sanitizing cabin could also be a conventional security cabin for access to a room requiring a security access, such as for example a bank or a jewelry store or a post office, which is modified in an obvious way for the one skilled in the art by adding: a unit 7 for sanitizing the person present inside the closed space delimited by said security cabin, and/or a unit 100I for measuring the temperature of the people who want to access said room, and/or a unit 100J for automatically detect the presence/absence of a mask on the face of the people who want to access said room, and/or a unit 100K for identifying the people who want to access said room, and/or a detecting device 200A-D adapted to count the people present in said room requiring a security access, where access is desired and to generate a monitoring signal relating to the number of people detected in the room, and at least one controller 12 of said unit 100I for the temperature measurement, and/or of the unit 100J for a mask presence/absence automatic detection, and/or of the unit 100K for identifying the person who wants to enter the cabin, and/or of the detecting device 200A-D, adapted to count the people present in a room 40 where access is desired;
wherein, preferably, the unit 100I for the temperature measurement has one or more of the features set forth in claims 2 and 5;
wherein, preferably, the unit 100J for a mask presence/absence automatic detection, has one or more of the features set forth in claim 3;
wherein, preferably, the unit 100K for identifying the people who want to enter said place has one or more of the features set forth in claims 4, 18 and 19;
wherein, preferably, the detecting device 200A-D, adapted to count the people present in said place 40 have one or more of the features set forth in claim 11;
wherein, preferably, the device 100Z for detecting the virus in a person, described below in detail, has one or more of the features set forth in claim 14.

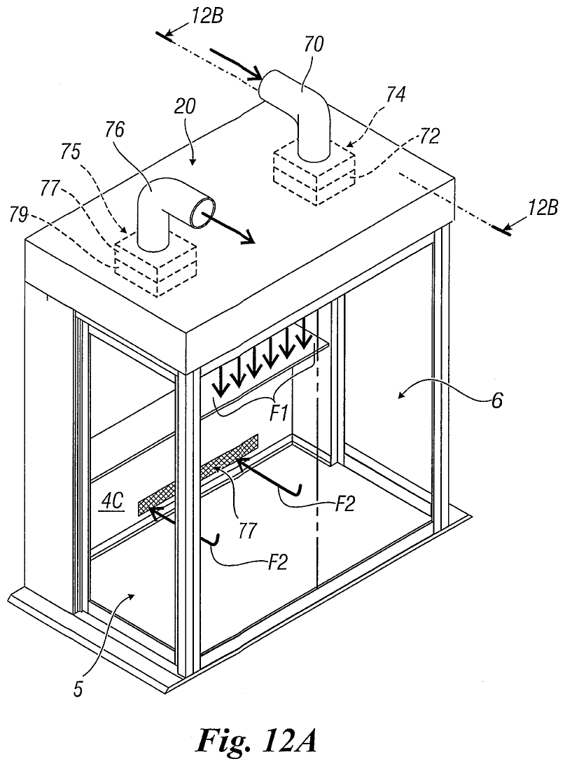

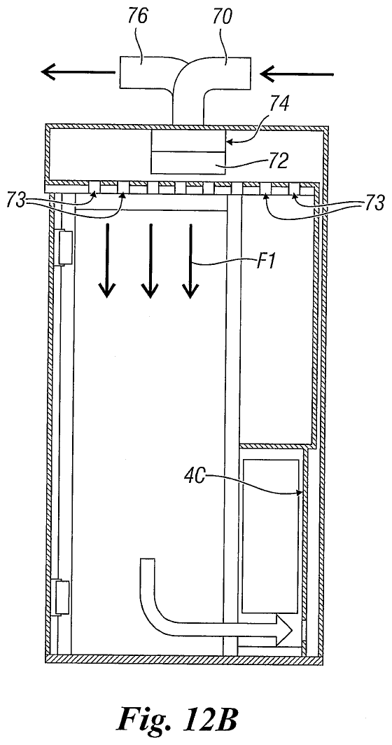

According to a further variant shown in FIGS. 12A and 12B, the cabin optionally also comprises a device 101Z (FIG. 12B) for the controlled air circulation inside the cabin 1, adapted in particular to create a negative pressure in the cabin.

The cabin according to this variant is preferably an air tight cabin, that is a cabin where a different pressure can be created, and preferably lower than the pressure outside the cabin itself; to this purpose, the various components of the cabin are specifically made for obtaining this effect, in any case in a conventional way for the one skilled ion the art, for example the doors 5 and 6 and the joining edges of the cabin wall have suitable seals of the conventional type for the one skilled in the art. The device 101Z (FIG. 12B) for the controlled air circulation inside the cabin comprise a circuit 74 for the air entrance in the cabin and a circuit 75 for the air exit from the cabin. The inlet circuit 74 comprises a room air intake mouth 70, one or more filters 71 for purifying the intake air, of the conventional type for the one skilled in the art, a device 72, for example of the compressor type, for sucking air and introducing it in the cabin at a predetermined first pressure P1, and a plurality of vents 73 for air inlet in the cabin which are preferably arranged in the cabin upper wall 2, so as to make air flow from above downwards as indicated by the arrows F1. The circuit 75 for the air outlet from the cabin comprises a plurality of vents 77 for the air outlet from the cabin which are preferably arranged in the lower part of the cabin, in correspondence with the wall parete 4C thereof, so as to suck air from the bottom as indicated by the arrows F2. Advantageously, the sucked air then passes into the crawl room of the walls 4C and reaches the upper part 20 of the cabin where a device 101Y is provided, for example of the compressor type, to suck the air from the cabin at a second pressure P2, preferably different and greater than the first pressure P1, and emit it from a mouth 76 for the outflow of air towards the external environment, and one or more filters 77 for the purification of the air sucked in from the cabin, of a conventional type for the one skilled in the art. The devices 101Z (FIG. 12B) for the air controlled circulation inside the cabin are preferably controlled by the controller 12, so as to create a negative pressure in the cabin, preferably at the end of the sanitation stage so as to suck in the air present in the cabin before the person gets out from the same cabin. According to a further variant, as shown in FIG. 10 the cabin also optionally comprises virus detecting device 100Z adapted to carry out a quick test on the person present in the cabin, to verify preferably by a blood drop, obtained preferably by fingertip pressure, if said person has come into contact with the virus and/or has an ongoing viral infection, in particular if he/she has produced antibodies, in particular immunoglobulins such as IgM and IgG, and therefore he/she has come into contact with a virus and/or has a an ongoing viral infection, in particular with the covid-19, wherein said devices 100Z are adapted to generate a monitoring signal Sm for the controller 12, the controller components (112, 113) of which for receiving and processing said virus monitoring signal are adapted to verify if the person has come into contact with a virus and/or has an ongoing viral infection, and to generate:
a first control signal, related to said verification, for said device 101C, 101A for automatically locking/unlocking the opening of the cabin 1 exit door 6, adapted to lock said exit door 8 if the person has come into contact with the virus and/or has an ongoing viral infection,
and/or to generate an information on the test result on an interface present in the cabin,
and/or to generate a signal for a remote supervision device 200.

These processes related to the virus verification are shown in the algorithm in FIG. 13J (wherein the optional or alternative functions discussed above have been represented within a dashed frame).

Advantageously, according to a further variant, in order to prevent a substance other than the desired and intended sanitizing substance from being introduced into the cabin by mistake, the circuit 11 for conveying the substance from the tank 8 to a plurality of emission members 10, provides after the tank 8, a device 101Y adapted to analyze the substance exiting from said tank and to verify that said substance has certain predetermined typical chemical and/or physical characteristics of the substance itself, that is that the substance is actually the one to be dispensed in the cabin. Said device 101Y is adapted to generate a substance monitoring signal relating to the detected chemical and/or physical characteristics, for the controller 12, the controller components 112, 113 of which for receiving and processing said monitoring signal are adapted to verify, on the basis of said monitoring signal, if the substances has the desired chemical and/or physical characteristics, and to generate a first control signal, related to said verification, for said device 101C, 101A for automatically locking/unlocking the opening of the cabin (1) entrance door 5, adapted to lock said entrance door 5 if the substance does not has the desired and predetermined chemical and/or physical characteristics and/or to generate an information relating to the verification result on an interface of the cabin, and/or to generate a signal for a remote supervision device 200.

Advantageously, according to a further variant, the cabin also has metal detector-type devices, adapted to detect the presence of metal objects on the person in the cabin, in particular weapons, and adapted to generate a signal for monitoring the presence of said metal objects for the process control and metal controller 12, the controller components 112, 113 of which for receiving and processing said monitoring signal are adapted to verify, on the basis of said monitoring signal, if the person has metal objects, and to generate a first control signal, related to said verification, for said device 101C, 101A for automatically locking/unlocking the opening of the cabin 1 exit door 6, adapted to lock said exit door 6 if the person has metal objects and/or to generate an information relating to the result of the verification on an interface of the cabin, and/or to generate a signal for a remote supervision device 200.

1) A system for sanitizing and controlling people who want to access a room (40), adapted to at least reduce the risk that people can bring pathogenic contaminants into said room (40), in particular viral pathogenic contaminants, such as for example coronavirus, comprising:
a sanitizing cabin (1) having a size such that it can house at least one person inside, comprising:
at least one upper wall (2), one lower wall (3) and at least one side wall (4A-D),
wherein said walls define a closed space (L) to which the access is possible through an entrance door (5) and from which it is possible to get out through an exit door (6), provided in the said walls in said at least one side wall (4A-D),
a sanitizing unit (7) for sanitizing the person present inside said closed space (L), comprising:
a tank (8) of a sanitizing substance,
a circuit (11) for conveying said substance from the tank to a plurality of emission members (10) emitting the substance in said closed space (L),
and at least one member (9) for automatically conveying said substance from said tank to said emission members through said supply circuit,
at least one controller (12) operatively connected to a sanitation activating element (32, 26, 100E), said controller (12) being configured to determine a process control command for said sanitizing unit (7) to supply the sanitizing substance to said closed room for a predetermined time, when it receives at least one activation signal from said sanitation activating element (32, 36, 100E).

2) A system according to claim 1, characterized in that:
it comprises a unit (100I) for automatically measuring the body temperature of the person who wants to enter the cabin (1), which is operatively connected to said controller (12),
said unit (100I) for automatically measuring the body temperature being configured for determining at least one monitoring signal (Sm) for said controller (12), related to the measured temperature,
at least the entrance door (5) of the cabin has a device (101C, 101A) for locking/unlocking the opening thereof,
wherein said controller (12) comprises an interface controller component (112) and a verification controller component (113) configured for receiving and processing said at least one temperature monitoring signal (Sm), adapted to verify whether the measured body temperature is higher than a predetermined limit value, and to generate at least one first control signal (Sc), related to said verification, for said device (101C, 101A) for automatically locking/unlocking the opening of the cabin (1) entrance door (5), adapted to lock said entrance door (5) if the measured temperature exceeds the predetermined temperature and vice versa to unlock the entrance door (5);
wherein, preferably, the unit (101I) for automatically measuring the body temperature of the person who wants to enter the cabin, comprises:
at least one thermal sensor (30), preferably two thermal sensors arranged at different heights, for example at one first high between 0.8 m and 1.30 m and a second high between 1.30 m and 1.90 m, at least one camera (32), and at least one proximity sensor (32A) adapted to calibrate the distance for the temperature instantaneous measurement and to activate a precise measuring by the thermal sensors,
or comprises at least one thermal scanner for instantaneously measuring the body temperature of the person present before the thermal scanner, which is operatively connected to said controller (12), to send the processing and control unit (12) at least one monitoring signal relating to the measured temperature.

3) A system according to claim 1, characterized in that:
it comprises a unit (100J) for automatically detecting the presence/absence of a protection masks on the face of the person who wants to enter the cabin (1), which is operatively connected to said controller (12),
wherein said unit (100J) for detecting the mask being configured for generating at least one mask presence/absence monitoring signal (Sm),
wherein said controller (12) comprises an interface controller component (112) and a verification controller component (113) configured for receiving and processing said at least one mask presence/absence monitoring signal and adapted to verify whether or not the person who wants to enter wears a mask on his/her face, and to generate at least one control signal related to said verification for at least another device (101C, 101A; 101F);
wherein said at least one other device (101C 101A; 101F) of the cabin (1) comprises:
a device (101C, 101A) for locking/unlocking the opening of said entrance door (5) the cabin,
said at least on control signal operates said device (101C, 101A) for locking/unlocking the entrance door (5) so as to automatically lock the entrance door (5) if a protection mask has not been detected on the face of the person who wants to enter the cabin;
or wherein said at least one other device (101C 101A; 101F) of the cabin (1) comprises a device (101D, 101B) for locking the opening of at least said exit door (6) of the cabin,
wherein said cabin has an internal protection mask housing device (22) for containing and supplying said masks,
wherein said at least one control signal operates said device (101D, 101B) for locking/unlocking the exit door (6) so as to automatically lock the opening of the exit door (5) if a protective mask has not been detected on the face of the person who wants to enter the cabin, but not to lock even said device (101C, 101A) for locking/unlocking the entrance door (5), so as to allow even a person on whose face a protection mask has not been detected to access the cabin; so that said person without the mask can withdraw a mask from said mask housing device (22) and can then get out of the cabin from the entrance door (5) to perform the check again on the presence of a protection mask of the face;
wherein preferably the unit (100J) for automatically detecting the mask on the person's face comprises: a video camera (32) and one or more proximity sensor (32A), and the verification controller component (113) preferably comprises: a further controller component (115) for analysing the images provided by said video camera (32),
wherein said further controller component (115) preferably comprises facial recognition controller component (115) adapted to process said images using artificial intelligence techniques and one or more neural networks.

4) A system according to claim 1, characterized in that it comprises:
an identification unit (100K) for identifying the person who wants to enter the cabin, which is operatively connected to said controller (12),
said person identification unit (100K) being configured to generate at least one identification signal;
wherein the controller (12) comprises an interface controller component (112) and a verification controller component (113) configured for receiving and processing said at least one identification signal, and adapted to generate at least one control signal related to said identification adapted to activate a plurality of devices (101 A-D, 101F 101G) of the cabin;
wherein said plurality of devices (101A-D, 101F, 101G) of the cabin comprise:
locking devices (100A, 10C) for locking the cabin entrance door (5),
and/or one first interface device (101F) provided outside the cabin preferably in correspondence with the entrance door (5), said first interface device being configured to receive said control signal for communicating a predetermined information, for example of the visual type, to inform the people who want to enter that the access is denied;
and/or a second interface device (101G) provided inside the cabin, said second interface device being configured, when it receives said control signal, to communicate customized information in the cabin, for example: promotional messages customized on the bases of the previously stored tastes of the identified person, or messages for directing the identified person to a fixed office, on the basis of the previously stored data of the identified person;
wherein said messages are stored in a memory controller component (118) of the controller (12);
wherein the identification unit (100K) for identifying the person who wants to enter the cabin, comprises at least one of the following devices: a magnetic stripe card readers, a bar code or Qr code reader, a RFID tag reader, a Bluetooth signal receiver, a video or photo camera, a keyboard or a microphone adapted to detect an acoustic message, a reader of the person's physical or biometric characteristics such as of the retina or iris or fingerprints.

5) A system according to claim 2, characterized in that the controller (12) comprises a controller component configured to process the signals received from the body temperature automatic measuring unit (101I) using artificial intelligence techniques and one or more neural networks;
and/or comprises a facial recognition controller component (115), preferably artificial intelligence algorithms, configured to recognize the person's face on the basis of the information received from a camera and configured to only identify and select the information on the temperature measured by the temperature sensors in correspondence with the face and not with other parts of the body, so as to get a high temperature measurement accuracy.

6) A system according to claim 1, characterized in that the sanitation activating element (32, 36, 100E) comprises:
a device (100G, H) for detecting the closing of the entrance and exit doors, operatively connected to the controller (12), and configured to generate at least one monitoring signal for said controller (12), related to the closure of the doors,
and/or a sensor member (100F) for detecting the presence of a person in the cabin (1), operatively connected to the controller (12) and configured to generate at least one monitoring signal for said controller (12) related to the presence of a person in the cabin,
and/or an operating member (32), for example of the button type, for starting sanitation adapted to be operated by the person present in the cabin, for example by means of a foot, operatively connected to the controller (12) and configured to generate at least one input sanitation start signal for said controller (12) related to a person's presence in the cabin,
wherein the controller (12) comprises controller components (112, 113) configured to receive and process said monitoring and/or start signals, to verify whether the doors are closed, and/or a person is present in the cabin, and/or the sanitation of the person in the cabin has been activated, and to generate at least one control signal related to said verification, for the conveying member (9) of the sanitation unit (7), so as to supply the substance to the substance supplying device (10) for a predetermined time.

7) A system according to claim 1, characterized in that the conveying member (9) for automatically conveying said substance from said tank (8) to said emission members (10) comprises a pump (9A) for moving a liquid sanitizing substance, a compressor (9B) to mix pressurized air to said liquid sanitizing substance, so that the said emission members (10) dispenses said mixture;
wherein preferably the emission members (10) are nebulizers (10A, B) adapted to dispense a nebulised sanitizing substance in the form of micro drops having sizes between 2 and 7 microns, more preferably equal to about 5 microns, which create a dry fog in the cabin which is not perceived as wet, given the sizes of the micro drops forming it;
wherein preferably the emission members (10) provide an inlet for the sanitizing substance that is liquid, and one entrance for the pressurized water, so as to dispense said substance nebulised and mixed with pressurized air;
wherein preferably the sanitizing substance is liquid substance based on hypochlorous acid, preferably of natural origin, more preferably 100% mineral, with neutral PH.

8) A system according to claim 1, characterized in that it provides an interface member (101G) inside the cabin comprising a screen, and/or a loudspeaker, for example a smart TV, and that the controller (12) is configured to generate control signals for said interface member (101G) so that it communicates to the person in the cabin predetermined messages, which are stored in a memory controller component (118) of the controller (12).

9) A system according to claim 1, characterized in that it provides at least one housing device (22), inside the cabin, for housing a plurality of individual protection devices, for example protection masks, and/or a hand sanitizing substance;
and in that said housing device (22) preferably comprises:
a device for automatically supply an individual protection device at a time, or a predetermined quantity of hand sanitizing substance, and a control device (22D) for said device for automatically supply an individual protection device at a time, or a predetermined quantity of hand sanitizing substance, operated by the person present in the cabin.

10) A system according to claim 1, characterized in that the cabin (1) has a size such that it can house at least one person in a wheelchair or a person with a shopping trolley inside it in said closed space (L),
and in that preferably the space (L) of the cabin (1) has the following sizes:
length (L1) at least 2000 mm, preferably between 2000 mm and 2500 mm, more preferably equal to about 2210 mm,
internal height (H1) at least 2100 mm, preferably between 2100 and 2300 mm, more preferably equal to about 2200 mm,
internal width (L2) at least 1000 mm, preferably between 1100 and 1300 mm, more preferably equal to about 1220 mm,
width (L3) of the two doors (5, 6) at least 800 mm, preferably between 800 and 900 mm, more preferably equal to about 850 mm.

11) A system according to claim 1, characterized in that:
it comprises a detecting device (200 A-D), adapted to automatically and continuously detect people present in said room (40), and to generate a monitoring signal (Sm) relating to the number of people detected in the room, for said controller (12) of the cabin,
wherein the controller (12) comprises an interface controller component (112) and a verification controller component (113) configured for receiving and processing said at least one people monitoring signal and adapted to verify whether the detected number of people is less or equal to a maximum number of people predetermined for said room, and to generate at least one first control signal related to this verified number of people, for a device (101C, 101A) for automatically locking/unlocking the opening of the cabin entrance door (5), so as to unlock said entrance door (5) if the number of people detected in the room is less than said maximum predetermined number and vice versa to lock said entrance door;
wherein preferably the detecting device (200 A-D), comprise at least one of the following devices:
electronic people-counting members (201), for example of the infrared ray type or photocell type or thermal or optical camera, arranged in correspondence with the access opening (41) and the exit opening (42) of the room (40),
and/or devices (202, 203) adapted to move people one person at a time and preferably also in one predetermined direction only, for example devices (202) of the turnstile type or devices (203) of the type having two movable bulkheads comprising known people-counting sensors;
wherein said people-counting detecting device (200A-D) are configured to provide the controller (12) with at least one signal relating to the detected number of people or related to said number of people;
and/or a plurality of video cameras (204), positioned in the room (40), which are operatively connected to a processing unit (205) configured to obtain the number of people present in the room from the signals generated by said plurality of video cameras, wherein said processing unit (205) preferably calculates the number of people present in the room at the same time, preferably through control systems that analyze the images generated by said video cameras and, for example, calculates the required number of people through artificial intelligence techniques and the use of neural networks.

12) A system according to claim 1, characterized in that the cabin (1) comprises a device (101Z) for the controlled air circulation inside the cabin (1), adapted in particular to create a negative pressure in the cabin so as to create a negative pressure, preferably at the end of the sanitation stage, so as to suck the air present in the cabin before the person gets out from the same cabin, and in that said device (101Z) for the controlled air circulation inside the cabin is operatively connected to the controller (12);
wherein preferably, the device (101Z) for the controlled air circulation inside the cabin comprise a circuit (74) for the air entrance in the cabin and a circuit (75) for the air exit from the cabin,
wherein said circuit for the air entrance (74) comprises a room air intake mouth (70), one or more filters (71) for purifying the intake air, a device (72) preferably of the compressor type, for sucking air and introducing it in the cabin at a predetermined first pressure (P1), and a plurality of vents (73) for air entrance in the cabin which are preferably arranged in the cabin upper wall (2), so as to make air flow from above downwards;
and wherein the circuit (75) for the air exit from the cabin comprises a plurality of vents (77) for the air exit from the cabin which are preferably arranged in the cabin lower wall, and a device (101Y), preferably of the compressor type, to suck the air from the cabin at a second predetermined pressure (P2), which is different and preferably greater than said first pressure (P1), and emit it from a mouth (76) for the outflow of air towards the external environment, and one or more filters (77) for the purification of the air sucked from the cabin.

13) A system according to claim 1, characterized in that the cabin (1) is an air tight cabin, that is a cabin where a different pressure can be created, and preferably lower than the pressure outside the cabin itself.

14) A system according to claim 1, characterized in that the cabin comprises a virus detecting device (100Z) for detecting a virus, preferably a covid-19 virus, adapted to carry out a quick test on the person present in the cabin to verify, in particular by a blood drop obtained preferably by fingertip pressure, if said person has come into contact with the virus and/or has an ongoing viral infection, in particular if he/she has produced antibodies, in particular immunoglobulins such as IgM and IgG, and therefore he/she has come into contact with a virus and/or has a an ongoing viral infection, in particular with the covid-19, wherein said devices (100Z) are operatively connected to the controller (12) and are configured for generating at least one monitoring signal (Sm) for said controller (12),
wherein said controller (12) comprises an interface controller component (112) and a verification controller component (113) configured for receiving and processing said at least one monitoring signal, and for verifying on the basis of said monitoring signal if the person has come into contact with a virus and/or has an ongoing viral infection, and to generate a first control signal, related to said verification, for a device (101C, 101A) for automatically locking/unlocking the opening of the cabin (1) exit door (6), adapted to lock said exit door (8) if the person has come into contact with the virus and/or has an ongoing viral infection, and/or to generate an information on the test result on an interface (101F) present in the cabin, and/or to generate a signal for a remote supervision device (200).

15) A system according to claim 1, characterized in that the cabin (1) has inside at least one cabinet (128) provided with an openable door (128A) for sanitizing objects belonging to the person which is inside the cabin, such as for example mobile phones, laptops, tablets, comprising an internal sanitation device (128B) for example of the type comprising UCV Lights.

16) A system according to claim 1, characterized in that sanitizing unit (7) comprises a detecting device (100D), preferably a load cell, to detect the fill level of the tank (8), said detecting device (100D) being operatively connected to the controller (12) and being configured for generating at least one monitoring signal relating to said tank filling level for the controller (12),
wherein said controller (12) comprises an interface controller component (112) and a verification controller component (113) configured for receiving and processing said level signal, adapted to evaluate the tank filling level, and to generate at least one control signal related to said evaluation for:
an interface (23) preferably provided outside the cabin at the entrance door (5), adapted to inform people who want to access the cabin that the cabin is no longer able to perform sanitization,
and/or for a locking device (101C, 101A) for locking the cabin entrance door (5), so as to lock the people access to the cabin,
and/or for a remote supervision device (24) so as to send said device an alarm signal.

17) A system according to claim 4, characterized in that the controller (12) provides an interface controller component (120) configured to connect to:
a system (400) for controlling the accesses of the place access to which is desired, and/or an automatic operator (401) of the place access to which is desired,
and/or a video surveillance system (4012) of the place access to which is desired.

18) A system according to claim 4, characterized in that the controller (12) provides a verification controller component (117) configured to verify whether the detected identification code is already present in a memory controller component (116) of the process and control and management controller (12), and whether one or more customized commands to be sent to the cabin control devices are associated to said identification code in said memory controller component (116),
wherein if said verification is positive the process and control and management controller (12) being configured for generating said customized command signals,
said customized commands for example comprising:
deactivation of the sanitizing unit to allow people passing through the cabin without activating sanitation; and/or
communicating particular customized messages via an interface (101F) outside the cabin or an interface (101G) inside the cabin, for example a smart TV.

19) A system according to claim 4, characterized in that the controller (12) provides a verification controller component (117) configured to verify whether the detected identification code is already present in the memory controller component (116) of the controller (12) and, if it is not present, it is memorized preferably together with the temporal data of access in said memory controller component (116), while if it is already present the temporal data of the required access are added to the data already present in said memory controller component (116).
